(19) United States
(12) Patent Application Publication
(10) Pub. No.: US 2003/0235543 A1
(43) Pub. Date: Dec. 25, 2003

(54) Topical administration of pharmacologically active bases for skin lightening

(76) Inventors: Howard I. Maibach, San Francisco, CA (US); Eric C. Luo, Plano, TX (US); Tsung-Min Hsu, San Diego, CA (US)

Correspondence Address: REED & EBERLE LLP, 800 MENLO AVENUE, SUITE 210, MENLO PARK, CA, 94025, US

(21) Appl. No.: 10/254,020

(22) Filed: Sep. 23, 2002

Related U.S. Application Data
(63) Continuation-in-part of application No. 10/178,079, filed on Jun. 21, 2002.

(51) Int. Cl. A61K 7/135

(52) U.S. Cl. 424/62

(57) ABSTRACT

Provided are methods and topical pharmaceutical formulations for skin lightening. The invention involves the topical administration of a pharmacologically active base in a formulation having a pH of about 8.0 to about 13.0, preferably about 8.0 to 11.5, more preferably about 8.5 to 11.5, and most preferably about 8.5 to 10.5. These basic formulations can be used to treat regions of hyperpigmented skin, comprising, as examples, age spots, freckles, disease-related hyperpigmented skin, melasma related to pregnancy or the use of oral contraceptives, and hyperpigmented skin due to chemical exposure or ingestion.

### CROSS-REFERENCE TO RELATED APPLICATIONS

This application is a continuation-in-part of U.S. Ser. No. 10/178,079, filed Jun. 21, 2002, which is hereby incorporated by reference.

### TECHNICAL FIELD

This invention relates generally to methods and pharmaceutical formulations for skin lightening, and relates more particularly to the use of a pharmacologically active base in such methods and formulations.

### BACKGROUND

Cosmetically disfiguring skin pigmentation irregularities and disorders are common and widespread, leading to a large demand for skin lightening products. The color of normal human skin is due primarily to melanin, hemoglobin, and carotenoids. Of these pigments, melanin is of particular importance in regard to skin pigmentation and cosmetology. The type and amount of melanin synthesized by the melanocyte (melanin-producing skin cell), and its distribution pattern in the surrounding keratinocytes, largely determine skin color. Two types of melanin are present in human skin: (1) eumelanin, which is the dark brown pigment found in most skin, hair, and eyes, and whose production is stimulated by exposure to ultraviolet light, and (2) pheomelanin, which is a yellow-orange pigment found mainly in the skin of very fair-skinned people, particularly those with red hair.

A basic understanding of the pigmentation pathway is helpful prior to the discussion of the skin-lightening agent used in conjunction with the method of the present invention. Melanin forms through a series of oxidative reactions involving the amino acid tyrosine in the presence of the enzyme tyrosinase. Tyrosinase converts tyrosine to dihydroxyphenylalanine (DOPA) and then to dopaquinone. Subsequently, dopaquinone is converted to dopachrome through auto-oxidation, and finally to dihydroxyindole or dihydroxyindole-2-carboxylic acid (DHICA) to form eumelanin. The latter reaction occurs in the presence of dopachrome tautomerase and DHICA oxidase. In the presence of cysteine or glutathione, dopaquinone is converted to cysteinyl DOPA or glutathione DOPA; subsequently, pheomelanin is formed.

Skin hyperpigmentation (or melasma) has a variety of causes, including exposure to ultraviolet (UV) light, genetic makeup, wounds, age (“age spots”), pregnancy (“mask of pregnancy” or “chloasma”), oral contraceptive use, exposure to certain chemicals, a number of skin and systemic diseases, and other factors. A safe and effective topical skin-lightening formulation is particularly useful for treating any such localized epidermal hyperpigmentation. Such a formulation is also useful for masking areas of skin hypopigmentation, such as in vitiligo or trauma-induced hypopigmentation, by lightening the surrounding skin. There is clearly a need for a safe and effective technique to reduce the degree of skin pigmentation in such cases.

Within the art of depigmentation, hydroquinone is one of the most widely prescribed agents worldwide. However, due to reports of potential mutagenicity and of widespread ochronosis in African nations, there has been an increasing impetus to find alternative herbal and pharmaceutical depigmenting agents. In fact, hydroquinone has been banned in Europe for use as a depigmenting agent. Other skin depigmentation agents, like azalaic acid, kojic acid, and certain -hydroxy acids, have been used, but all have shown varying levels of local irritation.

### BRIEF DESCRIPTION OF THE FIGURES

FIG. 1 is a graph comparing the in vitro release of protein from the stratum corneum upon incubation in a 0.05 M sodium hydroxide solution with that observed upon incubation in a glycolic, acid solution, as described in Example 1. As explained in Example 1, the experiment involved periodic evaluation over a 24-hour time period.

FIG. 2 is a graph illustrating the in vitro release of protein from the stratum corneum upon incubation for 10 minutes in glycolic acid and in 0.05 M solutions of different bases, as described in Example 2.

FIG. 3 is a graph illustrating the in vitro release of protein from the stratum corneum upon incubation in solutions of different bases (0.005 wt. % and 0.010 wt. %), at an incubation time of 40 minutes, as described in Example 3.

### SUMMARY OF THE INVENTION

It is accordingly a primary object of the invention to address the above needs in the art by providing a novel method and formulation for skin lightening.

It is another object of the invention to provide a method and pharmaceutical formulation for skin lightening, involving a topically applied formulation consisting essentially of a pharmacologically active base, at least one pharmaceutically acceptable topical carrier, and optionally at least one excipient, wherein the pharmacologically active base is present at a concentration effective to provide a pH within the range of approximately 8.0 to 13.0 at the skin surface, following topical application of the formulation. The formulation may be a lotion, cream, solution, paste, or the like, or may be contained in a skin patch comprised of a laminated composite intended for long-term adhesion to the body surface (typically throughout a delivery period in the range of about 8 to about 72 hours) in the region of the affected area.

### DETAILED DESCRIPTION OF THE INVENTION

### I. Definitions and Nomenclature

Before describing the present invention in detail, it is to be understood that this invention is not limited to particular formulation types, formulation components, manufacturing methods, or the like, as such may vary. It is also to be understood that the terminology used herein is for the purpose of describing particular embodiments only, and is not intended to be limiting.

It must be noted that, as used in this specification and the appended claims, the singular forms “a,” “an,” and “the” include plural referents unless the context clearly dictates otherwise.

The terms “pharmacologically active agent” and “-pharmacologically active base” are used interchangeably herein to refer to a pharmacologically effective basic compound or composition of matter which, when topically administered to a human patient, is effective to treat one or more aging-related skin conditions as defined above. Also included are derivatives and analogs of those compounds or classes of compounds specifically mentioned that also induce the desired effect, i.e., treatment of an aging-related skin condition.

The term “active agent” is used herein to refer to a chemical material or compound that induces a desired beneficial effect when administered topically, and include agents that are therapeutically and/or prophylactically effective as pharmaceuticals (“pharmacologically active agents”). Also included are derivatives and analogs of those compounds or classes of compounds specifically mentioned that also induce the desired effect. By an “effective” amount of an active agent is meant a nontoxic but sufficient amount of an active agent to provide the desired beneficial effect. More specifically, by a “therapeutically effective” or “prophylactically effective” amount is meant a nontoxic but sufficient amount of a beneficial agent to provide the desired therapeutic or prophylactic effect.

By “pharmaceutically acceptable,” such as in the recitation of a “pharmaceutically acceptable carrier,” or a “pharmaceutically acceptable derivative,” is meant a compound that is not biologically or otherwise undesirable, i.e., the compound may be incorporated into a pharmaceutical formulation of the invention and topically administered to a patient without causing any undesirable biological effects or interacting in a deleterious manner with any of the other components of the pharmaceutical formulation in which it is contained. The term “pharmaceutically acceptable” is used in an analogous manner, to refer to a compound or composition that may be incorporated into a pharmaceutical formulation herein (i.e., a pharmaceutical formulation containing one or more pharmacologically active agents) without causing undesirable biological effects or unwanted interaction with other components of the formulation.

The terms “treating” and “treatment” as used herein refer to reduction in severity and/or elimination of skin related conditions resulting from intrinsic and/or extrinsic aging processes of the skin. The present method of “treating” a skin condition related to aging, as the term is used herein, refers to the prevention of aging-related skin conditions as well as the treatment of aging-related skin conditions in affected individuals.

The terms “hyperpigmented region” and “affected area” are used interchangeably herein to mean a localized region having high melanin content. Examples include, but are not limited to, age spots, melasma, chloasma, freckles, disease-related hyperpigmentation, and post inflammatory pigmentation, sun-induced pigmented blemishes.

The term “skin lightening” as used herein means decreasing the pigment content of skin, usually the melanin content, including one or more of: overall lightening of basal skin tone, and lightening of hyperpigmented regions (melasma), including age spots, chloasma, freckles, disease-related hyperpigmentation, and post inflammatory hyperpigmentation of sun-induced pigmented blemishes.

The term “base” is used in its traditional sense, i.e., a substance that dissolves in water to produce hydroxide ions. The water is typically an aqueous fluid, and may be natural moisture at the skin surface. Similarly, any liquid or semisolid formulation that is used is preferably aqueous or used in conjunction with an overlayer of an occlusive material. Any base may be used provided that the compound provides free hydroxide ions in the presence of an aqueous fluid. Bases can provide free hydroxide ions either directly or indirectly and thus can also be referred to as “hydroxide-releasing agents.” Hydroxide-releasing agents that provide free hydroxide ions directly typically contain hydroxide groups and release the hydroxide ions directly into solution, for example, alkali metal hydroxides. Hydroxide-releasing agents that provide free hydroxide ions indirectly are typically those compounds that are acted upon chemically in an aqueous environment and the reaction produces hydroxide ions, for example metal carbonates or amines.

By an “effective” amount or a “therapeutically effective amount” of a pharmacologically active agent is meant a nontoxic but sufficient amount of the drug or agent to provide the desired effect, i.e., prevention or skin lightening treatment. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term “topical administration” is used in its conventional sense to mean topical application of a formulation to the skin.

The term “body surface” is used to refer to skin or mucosal tissue.

“Carriers” or “vehicles” as used herein refer to carrier materials suitable for incorporation in a topically applied composition. Carriers and vehicles useful herein include any such materials known in the art, which are nontoxic and do not interact with other components of the formulation in which it is contained in a deleterious manner.

The term “aqueous” refers to a formulation that contains water or that becomes water-containing following application to the skin or mucosal tissue.

In describing molecular structures and formulae herein, the phrase “having the formula” or “having the structure” is not intended to be limiting and is used in the same way that the term “comprising” is commonly used.

The term “alkyl” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to about 12 carbon atoms. The term “lower alkyl” intends an alkyl group of one to six carbon atoms, preferably one to four carbon atoms. “Substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the terms “alkyl” and “lower alkyl” include linear, branched, cyclic, unsubstituted, substituted, and/or heteroatom-containing alkyl or lower alkyl.

The term “alkenyl” as used herein refers to a branched or unbranched hydrocarbon group typically although not necessarily containing 2 to about 24 carbon atoms and at least one double bond, such as ethenyl, n-propenyl, isopropenyl, n-butenyl, isobutenyl, octenyl, decenyl, and the like. Generally, although again not necessarily, alkenyl groups herein contain 2 to about 12 carbon atoms. The term “lower alkenyl” intends an alkenyl group of two to six carbon atoms, preferably two to four carbon atoms. “Substituted alkenyl” refers to alkenyl substituted with one or more substituent groups, and the terms “heteroatom-containing alkenyl” and “heteroalkenyl” refer to alkenyl in which at least one carbon atom is replaced with a heteroatom.

The term “aryl” as used herein, and unless otherwise specified, refers to an aromatic substituent containing a single aromatic ring or multiple aromatic rings that are fused together, linked covalently, or linked to a common group such as a methylene or ethylene moiety. The common linking group may also be a carbonyl as in benzophenone, an oxygen atom as in diphenylether, or a nitrogen atom as in diphenylamine. Preferred aryl groups contain one aromatic ring and are referred to as “monocyclic aryl.” “Substituted aryl” refers to an aryl moiety substituted with one or more substituent groups, and the terms “heteroatom-containing aryl” and “heteroaryl” refer to aryl in which at least one carbon atom is replaced with a heteroatom.

The term “heteroatom-containing” as in a “heteroatom-containing hydrocarbyl group” refers to a molecule or molecular fragment in which one or more carbon atoms is replaced with an atom other than carbon, e.g., nitrogen, oxygen, sulfur, phosphorus or silicon. Similarly, the term “heteroalkyl” refers to an alkyl substituent that is heteroatom-containing, the term “heterocyclic” refers to a cyclic substituent that is heteroatom-containing, the term “heteroaryl” refers to an aryl substituent that is heteroatom-containing, and the like. When the term “heteroatom-containing” appears prior to a list of possible heteroatom-containing groups, it is intended that the term apply to every member of that group. That is, the phrase “heteroatom-containing alkyl, alkenyl, and alkynyl” is to be interpreted as “heteroatom-containing alkyl, heteroatom-containing alkenyl, and heteroatom-containing alkynyl.”

By “substituted” as in “substituted alkyl,” “substituted alkenyl,” “substituted aryl,” and the like, as alluded to in some of the aforementioned definitions, is meant that in the alkyl, alkenyl, aryl, or other moiety, at least one hydrogen atom bound to a carbon atom is replaced with one or more substituents that are functional groups such as hydroxyl, alkoxy, thio, amino, halo, and the like.

The terms “alkyl,” “alkenyl,” “aryl,” and the like are, unless otherwise indicated, intended to include unsubstituted, substituted, heteroatom-containing, and substituted heteroatom-containing such substituents.

### II. Indications

The invention pertains to a skin lightening treatment of an individual predisposed to or afflicted with hyperpigmentation, comprising topically administering to the affected skin area a pharmaceutical formulation that consists essentially of a pharmacologically active base, at least one pharmaceutically acceptable topical carrier, and optionally at least one excipient. Active agents in addition to the pharmacologically active base are unnecessary, as the base is itself quite effective in achieving skin lightening. Preferably, the base is present at a concentration sufficient to provide a pH at the region of the body surface within the range of about 8.0 to 13.0, preferably about 8.0 to 11.5, more preferably about 8.5 to 11.5, and most preferably about 8.5 to 10.5. In some aspects, the pH will be in the range of about 9.5 to 11.5, preferably 10.0 to about 11.5. In addition, or alternatively, the base is present at a concentration effective to provide a pH on the skin surface in the aforementioned ranges, following topical application of the formulation. The term “hyperpigmentation” includes a range of skin disorders, including, but not limited to, regional hyperpigmentations by melanocytic hyperactivity, such as idiopathic melasma arising during pregnancy (“mask of pregnancy” or “chloasma”) or as a consequence of oestrone/progestogen contraception; other localized hyperpigmentations by benign melanocytic hyperactivity and proliferation, such as senile pigmental blemishes; disease-related hyperpigmentation; accidental hyperpigmentations such as those due to photosensitization, genetic makeup, chemical ingestion or other exposure, age, and post-lesional scarring.

In a preferred embodiment, the hyperpigmentation that the method and formulation of the invention are used to treat is localized hyperpigmentation by benign melanocytic hyperactivity and proliferation, such as senile pigmental blemishes, also known as age spots. In a particularly preferred embodiment, the invention is used to treat age spots. As is well known, age spots are common among the elderly; they result from age-related, localized melanocytic hyperactivity.

In another preferred embodiment, the hyperpigmentation treated is a regional hyperpigmentation such as, for example, idiopathic melasma arising during pregnancy or as a consequence of oestrone/progestogen contraception.

In a further preferred embodiment, the hyperpigmentation that the method and formulation of the invention are used to treat is an accidental hyperpigmentation, e.g., a melanin disorder such as hyperpigmentation due to photosensitivity, hyperpigmentation due to genetic makeup, berloque dermatitis (resulting from exposure to oil of bergamot, specifically the bergapten, or 5-methoxypsoralens, contained therein), argyria (resulting from exposure to silver salts), or post-lesional scarring.

### III. The Pharmacologically Active Base

The pharmacologically active base of the invention is an inorganic or an organic pharmaceutically acceptable base. Preferred inorganic bases include inorganic hydroxides, inorganic oxides, inorganic salts of weak acids, and combinations thereof. Preferred organic bases are nitrogenous bases.

It has long been thought that strong bases, such as NaOH, were not suitable as pharmacologically active bases because they would damage skin. It has been now been discovered that the skin permeability of various drugs could be enhanced without skin damage by exposing the skin to a base or basic solution, in a skin contacting formulation or patch. The desired pH of the solution on the skin can be obtained using a variety of bases or base concentrations. Accordingly, the pH is selected so as to be low enough so as to not cause skin damage, but high enough to enhance skin permeation to various active agents. As such, it is important that the amount of base in any patch or formulation is optimized so as to increase the flux of the drug through the body surface while minimizing any possibility of skin damage. In general, this means that the pH at the body surface in contact with a formulation or drug delivery system of the invention is preferably in the range of approximately 8.0 to 13.0, preferably about 8.0 to 11.5, more preferably about 8.5 to 11.5 and most preferably about 8.5 to 10.5. In some aspects, the pH will be in the range of about 9.5 to 11.5, preferably 10.0 to about 11.5.

In one preferred embodiment, the pH at the body surface is the primary design consideration, i.e., the composition or system is designed so as to provide the desired pH at the body surface. Anhydrous formulations and transdermal systems may not have a measurable pH, and the formulation or system can be designed so as to provide a target pH at the body surface. Moisture from the body surface can migrate into the formulation or system, dissolve the base and thus release the base into solution, which will then provide the desired target pH at the skin's surface. In those instances, a hydrophilic composition is preferred. In addition, when using aqueous formulations, the pH of the formulation may change over time after it is applied on the skin. For example, gels, solutions, ointments, etc., may experience a net loss of moisture after being applied to the body surface, i.e., the amount of water lost is greater than the amount of water received from the body surface. In that case, the pH of the formulation may be different than its pH when manufactured. This problem can be easily remedied by designing the aqueous formulations to provide a target pH at the skin's surface.

In other embodiments of the invention, the pH of the formulation or the drug composition contained within a delivery system will be in the range of approximately 8.0 to 13.0, preferably about 8.0 to 11.5, more preferably about 8.5 to 11.5, and most preferably about 8.5 to 10.5. In some aspects, the pH will be in the range of about 9.5 to 11.5, preferably 10.0 to 11.5. In one embodiment of the invention the pH of the formulation is higher than the pH at the body surface. For example, if an aqueous formulation is used, moisture from the body surface can dilute the formulation, and thus provide for a different pH at the body surface, which will typically be lower than that of the formulation itself.

In one preferred embodiment, the body surface is exposed to a base or basic solution for a sufficient period of time so as to provide a high pH at the body surface, thus creating channels in the skin or mucosa for the drug to go through. It is expected that drug flux is proportional to the strength of the solution and the duration of exposure. However, it is desirable to balance the maximization of drug flux with the minimization of skin damage. This can be done in numerous ways. For example, the skin damage may be minimized by selecting a lower pH within the 8.0-13.0 range, by exposing the skin to the formulation or system for a shorter period of time, or by including at least one irritation-mitigating additive. Alternatively, the patient can be advised to change the location of application with each subsequent administration.

While certain preferred amounts are set forth below, it is understood that, for all of the inorganic and organic bases described herein, the optimum amount of any such base will depend on the strength or weakness of the base and its molecular weight, and other factors such as the number of ionizable sites in the active agent being administered and whether there are any acidic species present in the formulation or patch. One skilled in the art may readily determine the optimum amount for any particular base such that the degree of enhancement is optimized while the possibility of damage to the body surface is eliminated or at least substantially minimized.

Inorganic Base

Exemplary inorganic bases are inorganic hydroxides, inorganic oxides, inorganic salts of weak acids, and combinations thereof. Preferred inorganic bases are those whose aqueous solutions have a high pH, and are acceptable as food or pharmaceutical additives. Examples of such preferred inorganic bases are those listed below, along with their respective pHs. Some of the bases are identified in the form of a hydrate, and it is understood that when referring to a “base,” both the hydrated and non-hydrated forms are intended to be included.

| | pH of |
|---|---|
| Inorganic base | Aqueous Solution (concentration) |
| Ammonium hydroxide^{1,2,3} | 11.27 (1 N), 10.27 (0.001 N) |
| Sodium hydroxide^{1,2,3} | 14 (5%), 13 (0.5%), 12 (0.05%) |
| Potassium hydroxide^{1,2,3} | 13.5 (0.1 M) |
| Calcium hydroxide^{1,3} | 12.4 (saturated solution in water) |
| Magnesium hydroxide^{1,2,3} | 9.5 to 10.5 slurry |
| Magnesium oxide^{1,2,3} | 10.3 (saturated aqueous solution) |
| Calcium oxide³ | Soluble in water, Form Ca(OH)₂ |
| Sodium acetate^{1,3} | ˜8.9 (0.1 N) |
| Sodium acetate, trihydrate^{1,2} | 8.9 (0.1 N) |
| Sodium acetate, anhydrous^{1,2} | ˜8.9 (0.1 N) |
| Sodium borate decahydrate^{1,2} | ˜8.8-9.4, 9.15 to 9.2 (0.01 M) |
| Sodium borate^{1,2,3} | 8.8-9.4, 9.15 to 9.2 (0.01 M) |
| Sodium metaborate | Strongly alkaline |
| Sodium carbonate^{1,2,3} | ˜11.6 |
| Sodium carbonate hydrate¹ | ˜11.6 |
| Sodium carbonate anhydrous | ˜11.6 |
| Sodium bicarbonate^{1,2,3} | 8.3 (0.1 M fresh) |
| Sodium phosphate, tribasic^{1,3} | ˜11.5 (0.1%), ˜11.7 (0.5%), ˜11.9 |
| | (1.0%) |
| Sodium phosphate, tribasic | 11.5 (0.1%), 11.7 (0.5%), 11.9 |
| dodecahydrate | (1.0%) |
| Sodium phosphate, dibasic, | 9.1 (1%) |
| anhydrous^{1,2} | |
| Sodium phosphate, dibasic, | ˜9.5 |
| heptahydrate^{1,2} | |
| Sodium phosphate, dibasic^{1,3} | ˜9.5 |
| Sodium phosphate, dibasic, | ˜9.5 |
| dihydrate¹ | |
| Sodium phosphate, dibasic, | ˜9.5 |
| dodecahydrate | |
| Potassium carbonate^{1,3} | ˜11.6 |
| Potassium bicarbonate³ | 8.2 (0.1 M) |
| Potassium citrate^{1,2,3} | ˜8.5 |
| Potassium citrate monohydrate | ˜8.5 |
| Potassium acetate^{1,3} | 9.7 (0.1 M) |
| Potassium phosphate, dibasic^{1,2} | Aqueous solution is slightly alkaline |
| Potassium phosphate, tribasic³ | Aqueous solution is strongly alkaline |
| Ammonium phosphate, dibasic^{1,2,3} | ˜8 |

¹listed in the “Chemicals in Compliance with Pharmaceutical Standards: Inactive Ingredient Guide”
²listed in the “Handbook of Pharmaceutical Additives”
³listed in the FDA's food additive database

Inorganic Hydroxides

Inorganic hydroxides include, for example, ammonium hydroxide, alkali metal hydroxide and alkaline earth metal hydroxides, and mixtures thereof Preferred inorganic hydroxides include ammonium hydroxide; monovalent alkali metal hydroxides such as sodium hydroxide and potassium hydroxide; divalent alkali earth metal hydroxides such as calcium hydroxide and magnesium hydroxide; and combinations thereof.

The amount of inorganic hydroxide included in the compositions and systems of the invention, will typically represent about 0.3-7.0 wt %, preferably 0.5-4.0 wt %, more preferably about 0.5-3.0 wt %, most preferably about 0.75-2.0 wt %, of a topically applied formulation.

For formulations and systems containing chemical compounds that can be neutralized by or react with the inorganic base (i.e., acidic inactive ingredients), the amount of inorganic hydroxide is preferably the total of (1) the amount necessary to neutralize any such compounds (i.e., the “acidic species”), plus (2) about 0.3-7.0 wt %, preferably 0.5-4.0 wt %, more preferably about 0.5-3.0 wt %, most preferably about 0.75-2.0 wt %, of the formulation or system. As an example, for an acid addition salt, the base is preferably present in an amount just sufficient to neutralize the salt, plus an additional amount (i.e., about 0.3-7.0 wt %, preferably 0.5-4.0 wt %, more preferably about 0.5-3.0 wt %, most preferably about 0.75-2.0 wt %). Basic compounds in the form of a neutral, free base or a basic salt of an acidic compound are usually not affected by a base, and thus, for these compounds, the amount in (1) is usually the amount necessary to neutralize inactive components that are acidic. For delivery systems (i.e., “patches” to be applied to the skin), the aforementioned percentages are given relative to the total weight of the formulation components and the adhesive, gel, or liquid reservoir.

Inorganic Oxides

Inorganic oxides include, for example, magnesium oxide, calcium oxide, and the like.

The amount of inorganic oxide included in the compositions and systems of the invention may be substantially higher than the numbers set forth above for the inorganic hydroxide, and may be as high as 20 wt %, in some cases as high as 25 wt % or higher, but will generally be in the range of about 2-20 wt %. These amounts may be adjusted to take into consideration the presence of any base-neutralizable species.

Inorganic Salts of Weak Acids

Inorganic salts of weak acids include, ammonium phosphate (dibasic); alkali metal salts of weak acids such as sodium acetate, sodium borate, sodium metaborate, sodium carbonate, sodium bicarbonate, sodium phosphate (tribasic), sodium phosphate (dibasic), potassium carbonate, potassium bicarbonate, potassium citrate, potassium acetate, potassium phosphate (dibasic), potassium phosphate (tribasic); alkaline earth metal salts of weak acids such as magnesium phosphate and calcium phosphate; and the like, and combinations thereof.

Preferred inorganic salts of weak acids include, ammonium phosphate (dibasic) and alkali metal salts of weak acids.

The amount of any inorganic salt of a weak acid included in the compositions and systems of the invention may be substantially higher than the numbers set forth above for the inorganic hydroxide, and may be as high as 20 wt %, in some cases as high as 25 wt % or higher, but will generally be in the range of approximately 2-20 wt %. These amounts may be adjusted to take into consideration the presence of any base-neutralizable species.

Organic Bases

Organic bases suitable for use in the invention are nitrogenous compounds having an amino group, amido group, an oxime, a cyano group, an aromatic or non-aromatic nitrogen-containing heterocycle, a urea group, and combinations thereof. More specifically, examples of suitable organic bases are nitrogenous bases, which include, but are not limited to, primary amines, secondary amines, tertiary amines, amides, oximes, nitriles, aromatic and non-aromatic nitrogen-containing heterocycles, urea, and mixtures thereof. For nitrogenous bases, the amount of enhancing agent will typically represent about 0.5-4.0 wt %, preferably about 0.5-3.0 wt %, more preferably about 0.75-2.0 wt %, of the topically applied formulation. These amounts may be adjusted to take into consideration the presence of any base-neutralizable species. Still greater amounts of the nitrogenous base may be used, if desired, depending on the strength of the particular base.

Preferred organic bases are those whose aqueous solutions have a high pH or a high pKa (more preferably a pKa>9), and are acceptable as food or pharmaceutical additives. Examples of such preferred organic bases are those listed below.

| | pH of |
|---|---|
| Organic base | Aqueous Solution (concentration) |
| 2-amino-2-methyl-1,3-propanediol¹ | 10.8 (0.1 m) |
| 2-amino-2-methyl-1-propanol¹ | 11.3 (0.1 m) |
| Diethanolamine¹ | 11.0 (0.1 N) |
| Triethanolamine¹ | 10.5 (0.1 N) |
| Butylamine² | pKa = 10.56 |
| Dimethylamine² | Strong base, pKa = 10.73 |
| Cyclohexylamine² | Strong base, pKa = 10.64 |
| Ethylenediamine² | Strong base, pKa = 10.71 |
| Isopentylamine² | pKa = 10.6 |
| Monoethanolamine² | 12.1 (25%), 12.05 (0.1 N), pKa = 9.4 |
| Phenethylamine² | Strong base, pKa = 9.83 |
| Piperidine² | Strong base, pKa = 11.12 |
| Pyrrolidine² | Strong base, pKa = 11.27 |
| Trimethylamine² | Strong base, pKa = 9.81 |

¹listed in the “Handbook of Pharmaceutical Additives”
²listed in the FDA's food additive database

Suitable nitrogenous bases may contain any one or a combination of the following:

primary amino (—NH₂) groups;

mono-substituted (secondary) amino groups NHR where R is hydrocarbyl, generally either alkyl or aryl, e.g., lower alkyl or phenyl, and may be substituted with one or more nonhydrocarbyl substituents, e.g., 1 to 3 halo, hydroxyl, thiol, or lower alkoxy groups (such —NHR groups include, for example, methylamino, ethylamino, isopropylamino, butylamino, cyclopropylamino, cyclohexylamino, n-hexylamino, phenylamino, benzylamino, chloroethylamino, hydroxyethylamino, etc.);

di-substituted (tertiary) amino groups —NR^{a}R^{b} where R^{a} and R^{b} may be the same or different and are as defined above for R (suitable —NR^{a}R^{b} include, for example, dimethylamino, diethylamino, diisopropylamino, dibutylamino, methylpropylamino, methylhexylamino, methylcyclohexylamino, ethylcyclopropylamino, ethylchloroethylamino, methylbenzylamino, methylphenylamino, methyltoluylamino, methyl-p-chlorophenylamino, methylcyclohexylamino, etc.);

amides —(CO)—NR^{c}R^{d} where R^{c} and R^{d} may be the same or different and are either hydrogen or R, wherein R is as defined above (including, for example, amides wherein one of R^{c} and R^{d} is H and the other is methyl, butyl, benzyl, etc.);

cyano (—CN);

aromatic nitrogen-containing heterocycles, typically five- or six-membered monocyclic substituents, or bicyclic fused or linked five- or six-membered rings (such as pyrrolyl, pyrrolidinyl, pyridinyl, quinolinyl, indolyl, pyrimidinyl, imidazolyl, 1,2,4-triazolyl, tetrazolyl, etc.); and

non-aromatic nitrogen-containing heterocycles, typically four- to six-membered rings, including lactams and imides, e.g., pyrrolidino, morpholino, piperazino, piperidino, N-phenyl-propiolactam, -butyrolactam, -caprolactam, acetimide, phthalimide, succinimide, etc.

Primary amines, secondary amines, and tertiary amines may be generically grouped as encompassed by the molecular structure NR¹R²R³ wherein R¹, R², and R³ are selected from H, alkyl, hydroxyalkyl, alkoxyalkyl, alkenyl, hydroxyalkenyl, alkoxyalkenyl, cycloalkyl, cycloalkyl-substituted alkyl, monocyclic aryl, and monocyclic aryl-substituted alkyl, any of which, with the exception of H, may be substituted with at least one nonhydrogen substituent (e.g., halide or an additional amine moiety —NR¹R²), with the proviso that at least one of R¹, R², and R³ is other than H. Examples of such amines include, without limitation, diethanolamine, triethanolamine, isopropanolamine, triisopropanolamine, dibutanol amine, tributanol amine, N-dodecylethanolamine, N-(2-methoxyethyl) dodecylamine, N-(2,2-dimethoxyethyl)dodecylamine, N-ethyl-N-(dodecyl)ethanolamine, N-ethyl-N-(2-methoxyethyl)dodecylamine, N-ethyl-N-(2,2-dimethoxyethyl) dodecylamine, dimethyldodecylamine-N-oxide, monolauroyl lysine, dipalmitoyl lysine, dodecylamine, stearylamine, phenylethylamine, triethylamine, PEG-2 oleamine, PEG-5 oleamine, dodecyl 2-(N,N-dimethylamino)propionate, bis(2-hydroxyethyl)oleyl-amine, and combinations thereof.

Exemplary primary amines include 2-aminoethanol, 2-aminoheptane, 2-amino-2-methyl-1,3 propanediol, 2-amino-2-methyl-1-propanol, n-amylamine, benzylamine, 1,4-butanediamine, n-butylamine, cyclohexylamine, ethylamine, ethylenediamine, methylamine, α-methylbenzylamine, phenethylamine, propylamine, and tris(hydroxymethyl)aminomethane.

Exemplary secondary amines include compounds that contain groups such as methylamino, ethylamino, isopropylamino, butylamino, cyclopropylamino, cyclohexylamino, n-hexylamino, phenylamino, benzylamino, chloroethylamino, hydroxyethylamino, and so forth. Exemplary secondary amines include diethanolamine, diethylamine, diisopropylamine, and dimethylamine.

Exemplary tertiary amines include compounds that contain groups such as dibutylamino, diethylamino, dimethylamino, diisopropylamino, ethylchloroethylamino, ethylcyclopropylamino, methylhexylamino, methylcyclohexylamino, methylpropylamino, methylbenzylamino, methyl-p-chlorophenylamino, methylcyclohexylamino, methylphenylamino, methyltoluylamino, and so forth.

Exemplary tertiary amines include N,N-diethylaniline, N,N-dimethylglycine, triethanolamine, triethylamine, and trimethylamine. Amides, as will be appreciated by those in the art, have the molecular structure R⁴—(CO)—NR⁵R⁶ where R⁴, R⁵, and R⁶ are generally selected from H, alkyl, cycloalkyl, cycloalkyl-substituted alkyl, monocyclic aryl, and monocyclic aryl-substituted alkyl, any of which, with the exception of H, may be substituted with at least one nonhydrogen substituent as indicated with respect to amine compounds of formula NR¹R²R³. Examples of suitable amides herein include, without limitation, hexamethyleneacetamide, hexamethyleneoctamide, hexamethylene lauramide, hexamethylene palmitamide, N,N-dimethyl formamide, N,N-dimethyl acetamide, N,N-dimethyloctamide, N,N-dimethyldecamide, toluamide, dimethyl-m-toluamide, diethyl-m-toluamide, and combinations thereof.

Nitrogen-containing heterocycles suitable as the cosmeceutically active base herein include, by way of example, 2-pyrrolidone, 1-methyl-2-pyrrolidone, 5-methyl-2-pyrrolidone, 1,5-dimethyl-2-pyrrolidone, 1-ethyl-2-pyrrolidone, 1-propyl-3-dodecylpyrrolidine, 1-dodecyclazacycloheptan-2-one, ethylene thiourea, hydantoin, oxalylurea, imidazolidilyl urea, N-octadecyl morpholine, dodecylpyridinium, N-dodecylpyrrolidine, N-dodecylpiperidine, N-dodecylhomopiperidine, and combinations thereof.

For all cosmeceutically active bases herein, including both organic and inorganic bases, the optimum amount of any particular base will depend on the strength or weakness of the base, the molecular weight of the base, and other factors. One skilled in the art may readily determine the optimum amount of any particular base by ensuring that a formulation is effective to provide a pH at the skin surface, following application of the formulation, in the range of about 8.0-13.0, preferably about 8.0-11.5, more preferably about 8.5-11.5, and most preferably about 8.5-10.5. This in turn ensures that the degree of treatment is maximized while the possibility of damage to the skin is eliminated or at least substantially minimized.

For all pharmacologically active bases herein, including both organic and inorganic bases, the optimum amount of any particular agent will depend on the strength or weakness of the base, the molecular weight of the base, and other factors such as the number of ionizable sites in the drug administered and any other acidic species in the formulation or patch. One skilled in the art may readily determine the optimum amount for any particular agent by ensuring that a formulation is effective to provide a pH at the skin surface, following application of the formulation, in the range of about 8.0 to about 13.0, preferably about 8.0 to about 11.5, preferably in the range of about 8.5 to about 11.5, and most preferably about 8.5 to 10.5. This in turn ensures that the degree of treatment is maximized while the possibility of damage to the body surface is eliminated or at least substantially minimized.

### IV. Pharmaceutical Formulations and Skin Patches

The pharmaceutical formulation of the invention comprises a pharmaceutically acceptable topical carrier and an active agent that consists essentially of a pharmacologically active base.

The particular combination components in the formulation is determined in large part by chemical compatibility. That is, each component must coexist in the topical pharmaceutical formulation together with the base-without reacting or otherwise interacting with each other in a way that would diminish therapeutic efficacy or increase the likelihood of toxic or other adverse effects.

The formulation may be in any form suitable for application to the body surface, such as a cream, lotion, solution, gel, ointment, paste, plaster, paint, bioadhesive, or the like, and/or may be prepared so as to contain liposomes, micelles, and/or microspheres. For those formulations in which the base is an inorganic base, such as a hydroxide-releasing agent, it is preferred although not essential that water be present. Thus, such a formulation may be aqueous, i.e., contain water, or may be nonaqueous and optionally used in combination with an occlusive overlayer so that moisture evaporating from the body surface is maintained within the formulation upon application to the body surface and thereafter.

Formulations of the invention may optionally contain a pharmaceutically acceptable viscosity enhancer and/or film former. A viscosity enhancer increases the viscosity of the formulation so as to inhibit its spread beyond the site of application. Balsam Fir (Oregon) is an example of a pharmaceutically acceptable viscosity enhancer.

A film former, when it dries, forms a protective film over the site of application. The film inhibits removal of the active ingredient and keeps it in contact with the site being treated. An example of a film former that is suitable for use in this invention is Flexible Collodion, USP. As described in Remington: The Science and Practice of Pharmacy, 19th Ed. (Easton, PA: Mack Publishing Co., 1995), at page 1530, collodions are ethyl ether/ethanol solutions containing pyroxylin (a nitrocellulose) that evaporate to leave a film of pyroxylin. A film former may act additionally as a carrier. Solutions that dry to form a film are sometimes referred to as paints.

Creams, as also well known in the art, are viscous liquids or semisolid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

Lotions, which are preferred for the delivery of cosmetic agents, are preparations to be applied to the skin surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Solutions are homogeneous mixtures prepared by dissolving one or more chemical substances (solutes) in a liquid such that the molecules of the dissolved substance are dispersed among those of the solvent. The solution may contain other cosmeceutically acceptable chemicals to buffer, stabilize, or preserve the solute. Common examples of solvents used in preparing solutions are ethanol, water, propylene glycol, or any other cosmeceutically acceptable vehicles.

As is of course well known, gels are semisolid, suspension-type systems. Single-phase gels contain organic macromolecules distributed substantially uniformly throughout the carrier liquid, which is typically aqueous, but also, preferably, contains an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum.

Pastes are semisolid dosage forms in which the active agent is suspended in a suitable base. Depending on the nature of the base, pastes are divided between fatty pastes or those made from single-phase aqueous gels. The base in a fatty paste is generally petrolatum, hydrophilic petrolatum, or the like. The pastes made from single-phase aqueous gels generally incorporate carboxymethylcellulose or the like as a base.

Formulations may also be prepared with liposomes, micelles, and microspheres. Liposomes are microscopic vesicles having a lipid wall comprising a lipid bilayer, and, in the present context, encapsulate one or more components of the anti-aging formulations. Liposomal preparations herein include cationic (positively charged), anionic (negatively charged), and neutral preparations. Cationic liposomes are readily available. For example, N[1-2,3-dioleyloxy)propyl]-N,N,N-triethyl-ammonium (DOTMA) liposomes are available under the tradename Lipofectin® (GIBCO BRL, Grand Island, N.Y.). Similarly, anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), and dioleoylphoshatidyl ethanolamine (DOPE), among others. These materials can also be mixed with DOTMA in appropriate ratios. Methods for making liposomes using these materials are well known in the art.

Micelles are known in the art as comprised of surfactant molecules arranged so that their polar headgroups form an outer spherical shell, while the hydrophobic hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethylammonium chloride, dodecylammonium chloride, polyoxyl 8 dodecyl ether, polyoxyl 12 dodecyl ether, nonoxynol 10, and nonoxynol 30.

Microspheres, similarly, may be incorporated into the present formulations. Like liposomes and micelles, microspheres essentially encapsulate one or more components of the present formulations. Microspheres are generally, although not necessarily, formed from synthetic or naturally occurring biocompatible polymers, but may also be comprised of charged lipids such as phospholipids. Preparation of microspheres is well known in the art and described in the pertinent texts and literature.

Various additives, known to those skilled in the art, may be included in the topical formulations. For example, solvents, including relatively small amounts of alcohol, may be used to solubilize certain formulation components. Although the pharmacologically active bases herein do penetrate into the skin and have in fact been described as skin permeation enhancers, it may be desirable, with weaker bases or particularly severe dermatoses, to include an added permeation enhancer in the formulation. Examples of suitable enhancers include, but are not limited to, ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80) and lecithin (U.S. Pat. No. 4,783,450); alcohols such as ethanol, propanol, octanol, benzyl alcohol, and the like; polyethylene glycol and esters thereof such as polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly citric acid and succinic acid. Azone® and sulfoxides such as DMSO and C₁₀MSO may also be used, but are less preferred.

Most preferred enhancers are those lipophilic co-enhancers typically referred to as “plasticizing” enhancers, i.e., enhancers that have a molecular weight in the range of about 150 to 1000, with an aqueous solubility of less than about 1 wt. %, preferably less than about 0.5 wt. %, and most preferably less than about 0.2 wt. %. The Hildebrand solubility parameter of plasticizing enhancers is in the range of about 2.5 to about 10, preferably in the range of about 5 to about 10. Such enhancers are described in detail in co-pending, commonly assigned U.S. Patent application Ser. No. 09/738,410, filed on Dec. 14, 2000, and in International Patent Application No. PCT/US00/34483, published Jun. 21, 2001 as WO 01/43775 A2. Preferred lipophilic enhancers are fatty esters, fatty alcohols, and fatty ethers. Examples of specific and most preferred fatty acid esters include methyl laurate, ethyl oleate, propylene glycol monolaurate, propylene glycerol dilaurate, glycerol monolaurate, glycerol monooleate, isopropyl n-decanoate, and octyldodecyl myristate. Fatty alcohols include, for example, stearyl alcohol and oleyl alcohol, while fatty ethers include compounds wherein a diol or triol, preferably a C₂-C₄ alkane diol or triol, are substituted with one or two fatty ether substituents.

Additional permeation enhancers will be known to those of ordinary skill in the art of topical drug delivery, and/or are described in the pertinent texts and literature. See, e.g., Percutaneous Penetration Enhancers, Smith et al., editors (CRC Press, 1995).

The present formulations may also include conventional additives such as opacifiers, antioxidants, fragrance, colorant, gelling agents, thickening agents, stabilizers, surfactants, and the like. Other agents may also be added, such as antimicrobial agents to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

The formulations may also contain irritation-mitigating additives to minimize or eliminate the possibility of skin irritation or skin damage resulting from the pharmacologically active base or other components of the composition. Suitable irritation-mitigating additives include, for example: -tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritant-mitigating additive, if present, may be incorporated into the present formulations at a concentration effective to mitigate irritation or skin damage, typically representing not more than about 20 wt. %, more typically not more than about 5 wt. %, of the composition.

The pharmacologically active base may also be administered through the skin or mucosal tissue using a conventional skin patch, wherein the agent is contained within a laminated structure that serves as a drug delivery device to be affixed to the body surface. In such a structure, the pharmaceutical formulation is contained in a layer, or “reservoir,” underlying an upper backing layer. The laminated structure may contain a single reservoir, or it may contain multiple reservoirs.

In one embodiment, the reservoir comprises a polymeric matrix of a pharmaceutically acceptable adhesive material that serves to affix the system to the skin during drug delivery; typically, the adhesive material is a pressure-sensitive adhesive (PSA) that is suitable for long-term skin contact, and which should be physically and chemically compatible with the pharmacologically active base and any carriers, vehicles or other additives that are present. Examples of suitable adhesive materials include, but are not limited to, the following: polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene). Preferred adhesives are polyisobutylenes.

The backing layer functions as the primary structural element of the transdermal system and provides the device with flexibility and, preferably, occlusivity. The material used for the backing layer should be inert and incapable of absorbing drug, pharmacologically active base, or components of the formulation contained within the device. The backing is preferably comprised of a flexible elastomeric material that serves as a protective covering to prevent loss of drug and/or vehicle via transmission through the upper surface of the patch, and will preferably impart a degree of occlusivity to the system, such that the area of the body surface covered by the patch becomes hydrated during use. The material used for the backing layer should permit the device to follow the contours of the skin and be worn comfortably on areas of skin such as at joints or other points of flexure, which are normally subjected to mechanical strain, with little or no likelihood of the device disengaging from the skin due to differences in the flexibility or resiliency of the skin and the device. The materials used as the backing layer are either occlusive or permeable, as noted above, although occlusive backings are preferred, and are generally derived from synthetic polymers (e.g., polyester, polyethylene, polypropylene, polyurethane, polyvinylidine chloride, and polyether amide), natural polymers (e.g., cellulosic materials), or macroporous woven and nonwoven materials.

During storage and prior to use, the laminated structure includes a release liner. Immediately prior to use, this layer is removed from the device so that the system may be affixed to the skin. The release liner should be made from a drug/vehicle impermeable material, and is a disposable element that serves only to protect the device prior to application. Typically, the release liner is formed from a material impermeable to the pharmacologically active base, and which is easily stripped from the patch prior to use.

In an alternative embodiment, the active agent-containing reservoir and skin contact adhesive are present as separate and distinct layers, with the adhesive underlying the reservoir. In such a case, the reservoir may be a polymeric matrix as described above. Alternatively, the reservoir may be comprised of a liquid or semisolid formulation contained in a closed compartment or “pouch,” or it may be a hydrogel reservoir, or may take some other form. Hydrogel reservoirs are particularly preferred herein. As will be appreciated by those skilled in the art, hydrogels are macromolecular networks that absorb water and thus swell but do not dissolve in water. That is, hydrogels contain hydrophilic functional groups that provide for water absorption, but the hydrogels are comprised of crosslinked polymers that give rise to aqueous insolubility. Generally, then, hydrogels are comprised of crosslinked hydrophilic polymers such as a polyurethane, a polyvinyl alcohol, a polyacrylic acid, a polyoxyethylene, a polyvinylpyrrolidone, a poly(hydroxyethyl methacrylate) (poly(HEMA)), or a copolymer or mixture thereof. Particularly preferred hydrophilic polymers are copolymers of HEMA and polyvinylpyrrolidone.

Additional layers, e.g., intermediate fabric layers and/or rate-controlling membranes, may also be present in any of these drug delivery systems. Fabric layers may be used to facilitate fabrication of the device, while a rate-controlling membrane may be used to control the rate at which a component permeates out of the device. The component may be an active agent, an enhancer, or some other component contained in the drug delivery system. A rate-controlling membrane, if present, will be included in the system on the skin side of one or more of the drug reservoirs. The materials used to form such a membrane are selected to limit the flux of one or more components contained in the drug formulation. Representative materials useful for forming rate-controlling membranes include polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like.

Generally, the underlying surface of the transdermal device, i.e., the skin contact area, has an area in the range of about 5 cm² to 200 cm², preferably 5 cm² to 100 cm², more preferably 20 cm² to 60 cm². That area will vary, of course, with the size of the skin area to be treated, the amount of drug to be delivered, and the flux of the drug through the body surface. Larger patches will be necessary to accommodate larger treatment areas and larger quantities of active agent, while smaller patches can be used for smaller treatment areas and smaller quantities of active agent and/or for active agents that exhibit a relatively high permeation rate.

Such drug delivery systems may be fabricated using conventional coating and laminating techniques known in the art. For example, adhesive matrix systems can be prepared by casting a fluid admixture of adhesive, active agent, and vehicle onto the backing layer, followed by lamination of the release liner. Similarly, the adhesive mixture may be cast onto the release liner, followed by lamination of the backing layer. Alternatively, the drug reservoir may be prepared in the absence of drug or excipient, and then loaded by “soaking” in a drug/vehicle mixture. In general, these patches are fabricated by solvent evaporation, film casting, melt extrusion, thin film lamination, die cutting, or the like. The active agent will generally be incorporated into the device during patch manufacture rather than subsequent to preparation of the device.

In a preferred delivery system, an adhesive overlayer that also serves as a backing for the delivery system is used to better secure the patch to the body surface. This overlayer is sized such that it extends beyond the drug reservoir so that adhesive on the overlayer comes into contact with the body surface. The overlayer is useful because the adhesive/drug reservoir layer may lose its adhesion a few hours after application due to hydration. By incorporating such adhesive overlayer, the delivery system remains in place for the required period of time.

Other types and configurations of topically applied drug delivery systems may also be used in conjunction with the present invention, as will be appreciated by those skilled in the art of topical and transdermal drug delivery. See, for example, Ghosh, Transdermal and Topical Drug Delivery Systems (Interpharm Press, 1997), particularly Chapters 2 and 8.

### V. Administration

The method of delivery of the active agent may vary, but necessarily involves application of a formulation of the invention to an area of body surface for which skin lightening is desired, such as areas of hyperpigmentation. A cream, gel, lotion, or solution, for example, may be spread on the affected surface and gently rubbed in. A solution may be applied in the same way, but more typically will be applied with a dropper, swab, or the like, and carefully applied to the affected areas.

The dosage regimen will depend on a number of factors that may readily be determined, such as the severity of the hyperpigmentation and the responsiveness of the condition to be treated, but will normally be one or more doses per day, with a course of treatment lasting from several days to several months, or until a cure is effected or a diminution of pigmentation is achieved. One of ordinary skill may readily determine optimum dosages, dosing methodologies and repetition rates. In general, it is contemplated that the formulation will be applied one to four times daily. With a skin patch, the device is generally maintained in place on the body surface throughout a drug delivery period, typically in the range of 8 to 72 hours, and replaced as necessary.

The application regimen will depend on a number of factors that may readily be determined, such as the severity of the condition and its responsiveness to initial treatment, but will normally involve one or more applications per day on an ongoing basis. One of ordinary skill may readily determine the optimum amount of the formulation to be administered, administration methodologies and repetition rates. In general, it is contemplated that the formulations of the invention will be applied in the range of once or twice weekly up to once or twice daily.

It is to be understood that while the invention has been described in conjunction with the preferred specific embodiments thereof, the foregoing description is intended to illustrate and not limit the scope of the invention. Other aspects, advantages, and modifications will be apparent to those skilled in the art to which the invention pertains. Furthermore, the practice of the present invention will employ, unless otherwise indicated, conventional techniques of drug formulation, particularly topical and transdermal drug formulation, which are within the skill of the art. Such techniques are fully explained in the literature. See Remington: The Science and Practice of Pharmacy, cited supra, as well as Goodman & Gilman's The Pharmacological Basis of Therapeutics, 9th edition (New York: McGraw-Hill, 1996).

All patents, patent applications, and publications mentioned herein are hereby incorporated by reference in their entireties.

### EXPERIMENTAL

The practice of the present invention will employ, unless otherwise indicated, conventional techniques of pharmaceutical formulation and the like, which are within the skill of the art. Such techniques are fully explained in the literature. In the following examples, efforts have been made to ensure accuracy with respect to numbers used (e.g., amounts, temperatures, etc.) but some experimental error and deviation should be accounted for. Unless otherwise indicated, temperature is in degrees Celsius and pressure is at or near atmospheric pressure at sea level. All reagents were obtained commercially unless otherwise indicated.

### EXAMPLE 1

A topical gel of the invention is prepared by conventional pharmaceutical methods. The indicated amounts of the following ingredients are used:

| Ingredient | Amount | |
|---|---|---|
| Purified water | 600 | grams |
| Polyethylene glycol | 400 | grams |
| Potassium hydroxide | 0.01 | gram |
| Edetate disodium | 0.1 | gram |
| Carbomer 934P | 12.5 | grams |
| Poloxamer 407 | 2.0 | grams |
| Polysorbate 40 | 2.0 | grams |
| Butylated hydroxytoluene | 0.5 | grams |
| Benzyl alcohol | 10.0 | grams |

The carbomer 934P and the edetate disodium are added to 250 mL of the purified water, and the mixture is homogenized at low speed until the carbomer is dispersed. Next, the polaxamer 407, mixed with 250 mL of the purified water, is added to the carbomer mixture, and the resulting mixture is homogenized at low speed. The potassium hydroxide, dissolved in 100 mL of purified water, is added to this mixture, and the resulting mixture (Mixture 1) is homogenized at low speed. In a separate container, the polysorbate 40 and the butylated hydroxytoluene are added to the polyethylene glycol, and the resulting mixture is heated to 65° C. and maintained at this temperature until all the compounds are dissolved; this mixture is then allowed to cool to room temperature, at which time the benzyl alcohol is added, and the resulting mixture is homogenized at low speed. This mixture is then added to Mixture 1, and the resulting mixture is mixed at low speed until it is homogeneous, forming a gel of the invention.

### EXAMPLE 2

A topical cream of the invention is prepared by conventional pharmaceutical methods. The indicated amounts of the following ingredients are used:

| Ingredient | Amount | |
|---|---|---|
| Purified water | 370 | grams |
| White petrolatum | 250 | grams |
| Stearyl alcohol | 250 | grams |
| Propylene glycol | 120 | grams |
| Sodium lauryl sulfate | 10 | grams |
| Methylparaben | 0.25 | gram |
| Propylparaben | 0.15 | gram |
| Potassium hydroxide | 0.01 | gram |

The stearyl alcohol and the white petrolatum are melted together on a steam bath, and then maintained at a temperature of approximately 75° C. The other ingredients are then added, after previously having been dissolved in the purified water and warmed to 75° C., and the resulting mixture is stirred until it congeals into a cream of the invention.

### EXAMPLE 3

A skin patch of the invention may be prepared by conventional pharmaceutical methods. A square piece of sterile, finely woven gauze three centimeters on each side is placed in the center of a square piece of occlusive surgical adhesive tape five centimeters on each side. To the gauze is applied 0.4 mL of the gel of Example 1; the gel is allowed to soak into the gauze. This skin patch of the invention is used within three hours of preparation.

### EXAMPLE 4

The gel formulation of Example 1 is provided to patients having an area of skin hyperpigmentation. The area of skin hyperpigmentation is measured and photographed before treatment begins. Each of the patients is instructed to topically apply the formulation of the invention to the area of skin hyperpigmentation twice daily for eight weeks. The patients return to the clinic every seven days, when each area of skin hyperpigmentation is again measured and photographed. It is found that most of the areas of skin hyperpigmentation are significantly lightened after two weeks and that most have regained essentially normal skin color after eight weeks of treatment, with no scarring or other undesirable side effects.

### EXAMPLE 5

The skin patches of Example 3 are provided to patients having an area of skin hyperpigmentation. Each area of skin hyperpigmentation is measured and photographed before treatment begins. Each of the patients is instructed to topically apply a skin patch of the invention over the area of skin hyperpigmentation, replacing the old patch with a new one every 48 hours. The patients return to the clinic every seven days, when each area of skin hyperpigmentation is again measured and photographed. It is found that most of the areas of skin hyperpigmentation are significantly lightened after two weeks and that most have regained essentially normal skin color after eight weeks of treatment, with no scarring or other undesirable side effects.

### EXAMPLE 6

An in vitro keratolysis study was performed to compare the capability of glycolic acid and a base for removing protein from stratum corneum. Adult foot callus was collected, grounded and the portion between 50-80 mesh was used. Glycolic acid and sodium hydroxide solutions (concentration=0.05 M) were prepared in distilled water. Ten mg of ground callus powder was place in a test tube. Four ml of a test chemical was added to the callus and the test tube was placed in a shaker and incubated in a water bath at 37° C. for a predetermined incubation time. The incubation time periods were 10 min., 40 min, 2 hours, 4 hours, 6 hours and 24 hours. Six samples of each base were tested a total of six times each at each of the aforementioned incubation time periods.

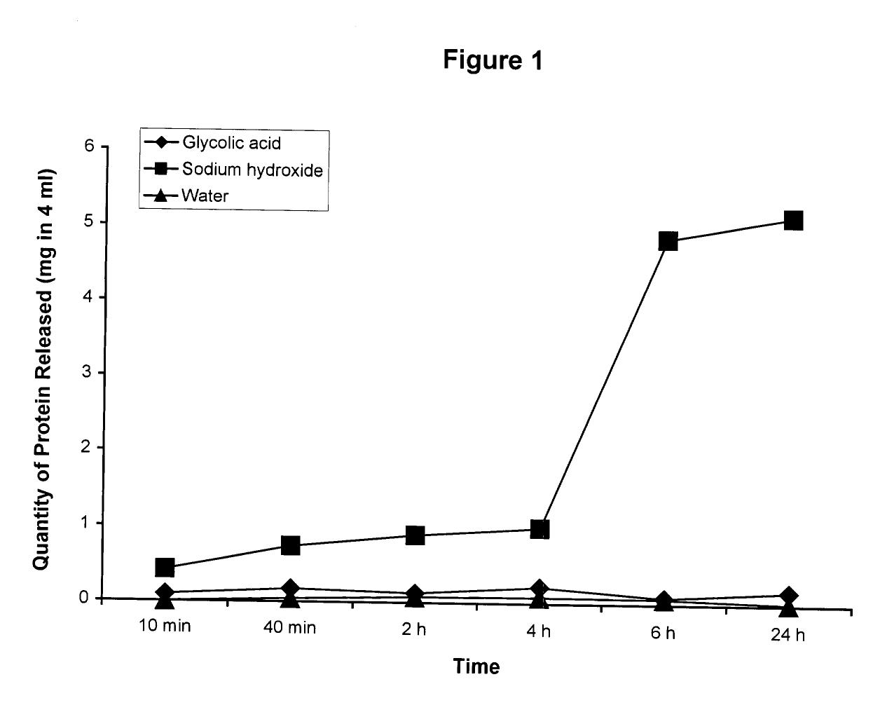

At the end of each incubation period, the test tube was removed from the shaker and the test tube was centrifuged. The solution was removed and filtered, and the protein in the solution was then determined using a BioRad® protein assay kit. The results are shown in the following Table 1 and FIG. 1.

TABLE 1
Quantity of Protein Released Using Different Bases and Incubation
Time Periods

| | Amount of Protein Released (mg/4 ml) | | | | | |
|---|---|---|---|---|---|---|
| | 10 min | 40 min | 2 hours | 4 hours | 6 hours | 24 hours |
| NaOH | 0.419 | 0.739 | 0.899 | 1.003 | 4.853 | 5.148 |
| Glycolic | 0.093 | 0.175 | 0.126 | 0.219 | 0.089 | 0.173 |
| Acid | | | | | | |
| Water | 0.002 | 0.043 | 0.074 | 0.086 | 0.073 | 0.021 |

The results indicated that at all time points 0.05 M NaOH solutions released more protein when compared to glycolic acid solutions at the same concentration and water control. However, glycolic acid did not show statistical differences in comparison with water. Sodium hydroxide solutions the amounts of protein released at 6 hours and 24 hours were significantly higher than the amounts released at 10 minutes, 40 minutes, 2 hours and 4 hours.

### EXAMPLE 7

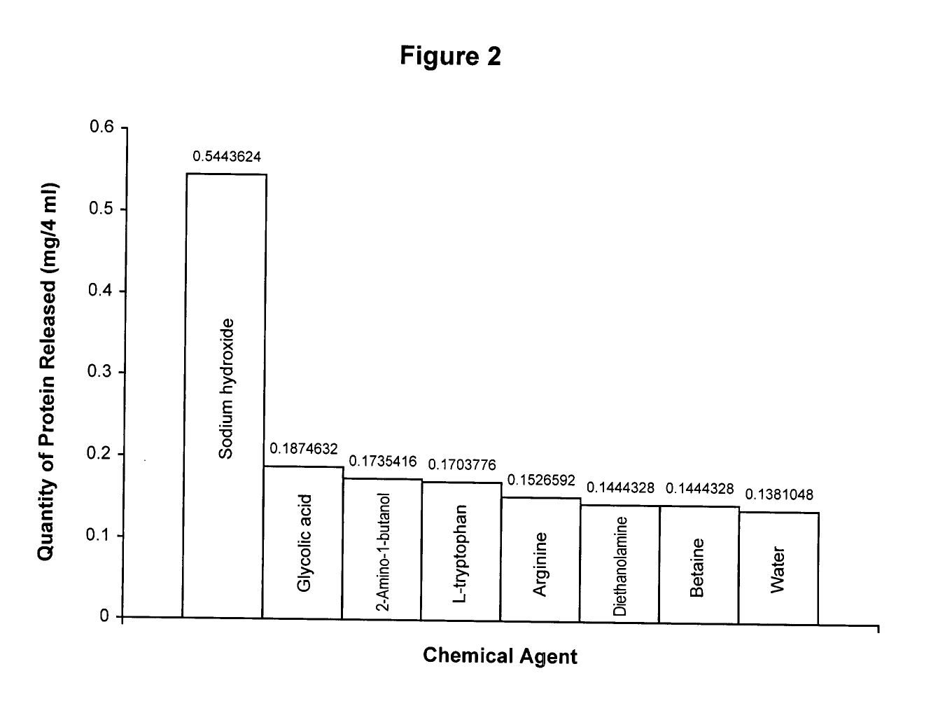

The procedure of Example 1 was then repeated to compare the capability of glycolic acid with respect to a number of different bases, including both inorganic and organic bases, with respect to removal of protein from stratum corneum. The results are shown in Table 2 (for the ten-minute incubation period) and FIG. 2.

TABLE 2
Quantity of Protein Released Using Different Bases
(Incubation Time = 10 minutes)

| | Amount of Protein Released (mg/4 ml) |
|---|---|
| NaOH | 0.544 |
| Glycolic Acid | 0.187 |
| Diethanolamine | 0.144 |
| L-tryptophan | 0.170 |
| Arginine | 0.153 |
| 2-amino-1-butanol | 0.174 |
| Betaine | 0.144 |
| Water | 0.138 |

The results indicated that 0.05 M NaOH solutions released more protein when compared to glycolic acid, other bases tested at the same concentration and water control. However, glycolic acid did and other bases did not show significant differences in comparison with water.

### EXAMPLE 8

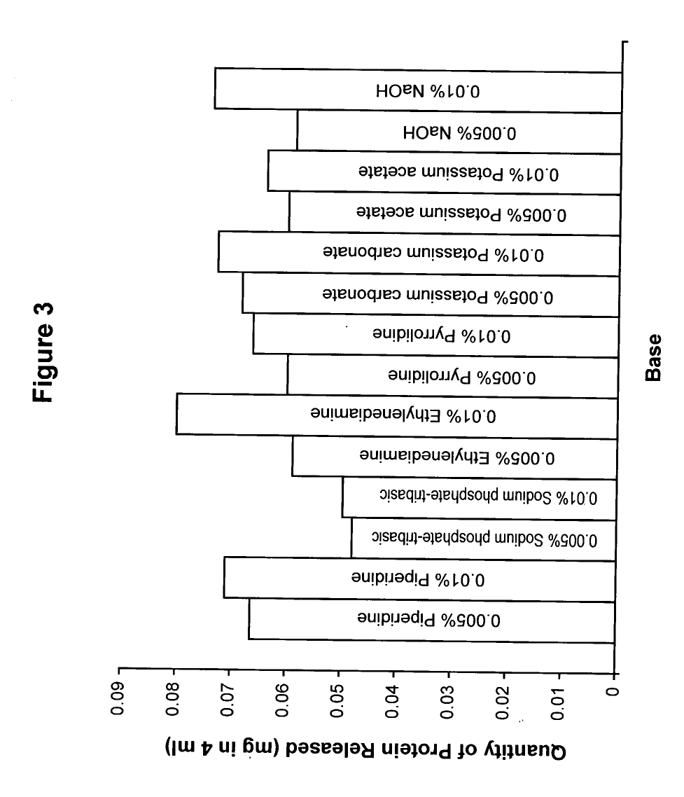

The procedure of Example 1 was then repeated with additional bases, all of which are listed in Table 3 (concentration=0.005% and 0.01%), using an incubation time period of 40 minutes. The results are shown in the following Table 3 and FIG. 3.

TABLE 3
Quantity of Protein Released Using Different Bases
(Incubating Time = 40 minutes)

| | Amount of Protein Released (mg/4 ml) | |
|---|---|---|
| | 0.005% | 0.01% |
| Piperidine | 0.066 | 0.071 |
| Sodium phosphate-tribasic | 0.048 | 0.050 |
| Ethylenediamine | 0.059 | 0.080 |
| Pyrrolidine | 0.060 | 0.066 |
| Potassium Carbonate | 0.068 | 0.073 |
| Potassium Acetate | 0.060 | 0.064 |
| NaOH | 0.059 | 0.074 |

The results indicate that solutions of bases listed in Table 3 released about the same amounts of protein at 0.005% and 0.01% concentrations. The quantity of protein released was very small when compared to the data obtained in Examples 1 and 2 at a higher concentration of base.

### EXAMPLE 9

To determine whether the base-containing compositions of Examples 1 through 5 or related compositions (“compositions”) are therapeutically effective for alleviating or improving skin pigmentation irregularities, patients and voluntary subjects (collectively “participants”) are included in a study where the compositions are topically administered twice daily on the face and hands of the participants for a time period of 6 to 12 months. For optimal bioavailability, the compositions are formulated to contain 5 to 30%, and preferably between 8 to 20% of the active agent. For the duration of the study, the participants are instructed to avoid sun exposure, or if such is not possible, to use a sunscreen product with a sun protection factor of 15 or greater.

Prior to attending the study, each participant is requested to appear to the study without any make-up or concealers on their face or hands. At the beginning of the study, the quality of the participants skin is examined and documented. Skin characteristics that should be noted are color, tone, texture (i.e., smooth, rough), translucency (glossy, dull), and touch (soft, coarse). Skin characteristics that should be carefully documented are areas showing hyperpigmentation such as age spots, freckles, blemishes, etc. In addition to the examination, four photographs are taken of each participant: one of each side of the face and one of the back of each hand. To take the photographs, the same light source and two foot distance is used to photograph the face and hands of each participant. For photographs of the face, the camera is aimed perpendicularly at a locus on the frontal aspect of each cheek. For photographs of the hands, the camera is aimed perpendicularly at the centermost part of the back of each hand.

For the duration of the study, participants are to return to the clinic conducting the study on a monthly basis for follow-up examinations and photographs. Participants are instructed to continue to not wear any make-up or concealers on their face and hands when appearing for their monthly photographs. During examination, the quality of the participants' skin is examined in order to see how the skin has reacted to the topical base, i.e., whether the hyperpigmented areas of skin have become less noticeable; whether the skin color and tone has become more uniform; whether the skin has become smoother, glossier, or softer; and whether the skin has become irritated. If a participant shows signs of irritation from the topical composition applied, such should be recorded, and a lower concentration of the same composition should be administered for the following month. If the participant continues to show signs of irritation with the lower concentration compositions, then a different composition should be administered to the patient until no more irritation is observed. All participants are instructed to notify the clinic immediately and to discontinue use of the compositions if the irritation becomes severe. Severe irritation will be a subjective determination but will always involve irritation causing extreme discomfort. Examples of severe irritation may include symptoms such as visible redness, itching, dryness, and flaking of the skin that continues without interruption for a period of ten days or more.

### EXAMPLE 10

Compositions containing a high concentration (50% or higher) to full-strength (100%) of the bases of Examples 1, 2, and 4 or related compositions (compositions) may be used in an office procedure or treatment. The compositions are topically applied to the patient's skin by a dermatologist or a trained professional and, using rubber gloves, gently massaged in with the fingers or a cotton ball. The length of time of the massage will depend on the strength of the composition and the patient's skin sensitivity to the composition, but usually will be for one to a few minutes. After the massage, the patient's skin is gently rinsed with water.

For optimal effect, the office procedure should be repeated every two to three weeks. Photographs, as described in the previous example, should be taken of the patient's skin before the first application and repeated before every visit or before every second or third visit. Improvements should be seen after three to five office visits; however, with some patients, eight to twelve office visits may be necessary.

If so desired, the home treatment regime set forth in the prior example may be combined with the office procedure set forth herein to increase the therapeutic effects of the base-containing composition. Under this procedure, the patient is to apply the lower dose compositions or the skin patches as set forth in Example 9 between office visits.

We claim:

1. A method of lightening the skin of an individual afflicted with hyperpigmentation, comprising topically applying to an affected area of the individual's skin a formulation consisting essentially of a pharmacologically active base, at least one pharmaceutically acceptable carrier, and optionally at least one excipient.

2. The method of claim 1, wherein the hyperpigmentation is selected from the group consisting of keratoses, age spots, idiopathic melasmas, chloasma, hyperpigmentation resulting as a consequence of chemical ingestion or other exposure, hyperpigmentation due to photosensitivity, hyperpigmentation due to genetic makeup, disease-related hyperpigmentation, post-lesional scarring, berloque dermatitis, and argyria.

3. The method of claim 1, wherein the pharmacologically active base is present in the formulation at a concentration effective to provide a formulation pH in the range of approximately 8.0 to 13.0.

4. The method of claim 1, wherein the pharmacologically active base is present in the formulation at a concentration effective to provide a pH on the skin surface, following topical application of the formulation, in the range of approximately 8.0 to 13.0.

5. The method of claim 3, wherein the pH is in the range of approximately 8.0 to 11.5.

6. The method of claim 3, wherein the pH is in the range of approximately 8.5 to 11.5.

7. The method of claim 3, wherein the pH is in the range of approximately 8.5 to 10.5.

8. The method of claim 1, wherein the formulation is aqueous.

9. The method of claim 8, wherein the aqueous formulation is a cream.

10. The method of claim 8, wherein the aqueous formulation is a gel.

11. The method of claim 8, wherein the aqueous formulation is a lotion.

12. The method of claim 8, wherein the aqueous formulation is a solution.

13. The method of claim 1, wherein the pharmacologically active base is an inorganic base.

14. The method of claim 13, wherein the pharmacologically active base is selected from the group consisting of inorganic hydroxides, inorganic oxides, metal salts of weak acids, and mixtures thereof.

15. The method of claim 14, wherein the pharmacologically active base is an inorganic hydroxide.

16. The method of claim 15, wherein the inorganic hydroxide is selected from the group consisting of ammonium hydroxide, alkali metal hydroxides, alkaline earth metal hydroxides, and mixtures thereof.

17. The method of claim 16, wherein the inorganic hydroxide is selected from the group consisting of ammonium hydroxide, sodium hydroxide, calcium hydroxide, potassium hydroxide, magnesium hydroxide, and mixtures thereof.

18. The method of claim 17, wherein the inorganic hydroxide is sodium hydroxide.

19. The method of claim 13, wherein the base is an inorganic oxide.

20. The method of claim 13, wherein the base is a metal salt of a weak acid.

21. The method of claim 1, wherein the pharmacologically active base is a nitrogenous base.

22. The method of claim 1, wherein the pharmacologically active base is an organic base.

23. The method of claim 22, wherein the organic base is selected from primary amines, secondary amines, tertiary amines, amides, oximes, nitrites, nitrogen-containing heterocycles, and urea.

24. The method of claim 23, wherein the organic base is a primary amine, a secondary amine, or a tertiary amine.

25. The method of claim 24, wherein the organic base has the structure NR¹R²R³ wherein R¹, R² and R³ are selected from H, alkyl, hydroxyalkyl, alkoxyalkyl, alkenyl, hydroxyalkenyl, alkoxyalkenyl, cycloalkyl, cycloalkyl-substituted alkyl, monocyclic aryl, and monocyclic aryl-substituted alkyl, with the proviso that at least one of R¹, R² and R³ is other than H.

26. The method of claim 24, wherein the organic base is selected from the group consisting of diethanolamine, triethanolamine, isopropanolamine, triisopropanolamine, dibutanol amine, tributanol amine, N-dodecylethanolamine, N-(2-methoxyethyl) dodecylamine, N-(2,2-dimethoxyethyl)dodecylamine, N-ethyl-N-(dodecyl)ethanolamine, N-ethyl-N-(2-methoxyethyl)dodecylamine, N-ethyl-N-(2,2-dimethoxyethyl) dodecylamine, dimethyldodecylamine-N-oxide, monolauroyl lysine, dipalmitoyl lysine, dodecylamine, stearylamine, phenylethylamine, triethylamine, PEG-2 oleamine, PEG-5 oleamine, dodecyl 2-(N,N-dimethylamino)propionate, bis(2-hydroxyethyl)oleylamine, and combinations thereof.

27. The method of claim 23, wherein the organic base is an amide.

28. The method of claim 24, wherein the amide has the structure R⁴—(CO)—NR⁵R⁶ where R⁴, R⁵ and R⁶ are independently selected from H, alkyl, cycloalkyl, cycloalkyl-substituted alkyl, monocyclic aryl, and monocyclic aryl-substituted alkyl.

29. The method of claim 28, wherein the amide is selected from the group consisting of hexamethyleneacetamide, hexamethyleneoctamide, hexamethylene lauramide, hexamethylene palmitamide, N,N-dimethyl formamide, N,N-dimethyl acetamide, N,N-dimethyloctamide, N,N-dimethyldecamide, toluamide, dimethyl-m-toluamide, diethyl-m-toluamide, and combinations thereof.

30. The method of claim 23, wherein the organic base is a nitrogen-containing heterocycle.

31. The method of claim 30, wherein the nitrogen-containing heterocycle is selected from the group consisting of 2-pyrrolidone, 1-methyl-2-pyrrolidone, 5-methyl-2-pyrrolidone, 1,5-dimethyl-2-pyrrolidone, 1-ethyl-2-pyrrolidone, 1-propyl-3-dodecylpyrrolidine, 1-dodecyclazacycloheptan-2-one, ethylene thiourea, hydantoin, oxalylurea, imidazolidilyl urea, N-octadecyl morpholine, dodecylpyridinium, N-dodecylpyrrolidine, N-dodecylpiperidine, N-dodecylhomopiperidine, and combinations thereof.

32. The method of claim 1, wherein the formulation is applied periodically over an extended time period.

33. The method of claim 1, wherein the formulation is applied approximately twice weekly.

34. The method of claim 1, wherein the formulation is applied once daily.

35. The method of claim 1, wherein the formulation is applied twice daily.

36. The method of claim 1, wherein the formulation is applied on an as-needed basis.

37. The method of claim 31, wherein said extended time period is at least three months.

38. The method of claim 36, wherein said extended time period is at least four months.

39. A topical formulation for lightening the skin of an individual afflicted with hyperpigmentation, consisting essentially of a pharmacologically active base, at least one pharmaceutically acceptable topical carrier, and optionally at least one excipient, wherein the active agent is agent is present at a concentration sufficient to provide a formulation pH in the range of approximately 8.0 to 13.0.

40. The formulation of claim 39, wherein the pH is in the range of approximately 8.0 to 11.5.

41. The formulation of claim 40, wherein the pH is in the range of approximately 8.5 to 11.5.

42. The formulation of claim 41, wherein the pH is in the range of approximately 8.5 to 10.5.

43. The formulation of claim 39, wherein the carrier is aqueous.

44. The formulation of claim 43, selected from the group consisting of a cream, a gel, a lotion, and a paste.

45. The formulation of claim 44, in the form of a cream.

46. The formulation of claim 44, in the form of a gel.

47. The formulation of claim 44, in the form of a lotion.

48. The formulation of claim 44, in the form of a solution.

49. The formulation of claim 39, wherein the pharmacologically active base is a hydroxide-releasing agent.

50. The formulation of claim 49, wherein the pharmacologically active base is selected from the group consisting of inorganic hydroxides, inorganic oxides, metal salts of weak acids, and mixtures thereof.

51. The formulation of claim 50, wherein the pharmacologically active base is an inorganic hydroxide.

52. The formulation of claim 51, wherein the inorganic hydroxide is selected from the group consisting of ammonium hydroxide, alkali metal hydroxides, alkaline earth metal hydroxides, and mixtures thereof.

53. The formulation of claim 52, wherein the inorganic hydroxide is selected from the group consisting of ammonium hydroxide, sodium hydroxide, calcium hydroxide, potassium hydroxide, magnesium hydroxide, and mixtures thereof.

54. The formulation of claim 53, wherein the inorganic hydroxide is sodium hydroxide.

55. The formulation of claim 50, wherein the base is an inorganic oxide.

56. The formulation of claim 50, wherein the base is a metal salt of a weak acid.

57. The formulation of claim 39, wherein the pharmacologically active base is a nitrogenous base.

58. The formulation of claim 39, wherein the pharmacologically active base is an organic base.

59. The formulation of claim 58, wherein the organic base is selected from primary amines, secondary amines, tertiary amines, amides, oximes, nitrogen-containing heterocycles, and urea.

60. The formulation of claim 59, wherein the organic base is a primary amine, a secondary amine, or a tertiary amine.

61. The formulation of claim 60, wherein the organic base has the structure NR¹R²R³ wherein R¹, R² and R³ are selected from H, alkyl, hydroxyalkyl, alkoxyalkyl, alkenyl, hydroxyalkenyl, alkoxyalkenyl, cycloalkyl, cycloalkyl-substituted alkyl, monocyclic aryl, and monocyclic aryl-substituted alkyl, with the proviso that at least one of R¹, R² and R³ is other than H.

62. The formulation of claim 60, wherein the organic base is selected from the group consisting of diethanolamine, triethanolamine, isopropanolamine, triisopropanolamine, dibutanol amine, tributanol amine, N-dodecylethanolamine, N-(2-methoxyethyl) dodecylamine, N-(2,2-dimethoxyethyl)dodecylamine, N-ethyl-N-(dodecyl)ethanolamine, N-ethyl-N-(2-methoxyethyl)dodecylamine, N-ethyl-N-(2,2-dimethoxyethyl) dodecylamine, dimethyldodecylamine-N-oxide, monolauroyl lysine, dipalmitoyl lysine, dodecylamine, stearylamine, phenylethylamine, triethylamine, PEG-2 oleamine, PEG-5 oleamine, dodecyl 2-(N,N-dimethylamino)propionate, bis(2-hydroxyethyl)oleylamine, and combinations thereof.

63. The formulation of claim 59, wherein the organic base is an amide.

64. The formulation of claim 63, wherein the amide has the structure R⁴—(CO)—NR⁵R⁶ where R⁴, R⁵ and R⁶ are independently selected from H, alkyl, cycloalkyl, cycloalkyl-substituted alkyl, monocyclic aryl, and monocyclic aryl-substituted alkyl.

65. The formulation of claim 64, wherein the amide is selected from the group consisting of hexamethyleneacetamide, hexamethyleneoctamide, hexamethylene lauramide, hexamethylene palmitamide, N,N-dimethyl formamide, N,N-dimethyl acetamide, N,N-dimethyloctamide, N,N-dimethyldecamide, toluamide, dimethyl-m-toluamide, diethyl-m-toluamide, and combinations thereof.

66. The formulation of claim 59, wherein the organic base is a nitrogen-containing heterocycle.

67. The formulation of claim 64, wherein the nitrogen-containing heterocycle is selected from the group consisting of 2-pyrrolidone, 1-methyl-2-pyrrolidone, 5-methyl-2-pyrrolidone, 1,5-dimethyl-2-pyrrolidone, 1-ethyl-2-pyrrolidone, 1-propyl-3-dodecylpyrrolidine, 1-dodecyclazacycloheptan-2-one, ethylene thiourea, hydantoin, oxalylurea, imidazolidilyl urea, N-octadecyl morpholine, dodecylpyridinium, N-dodecylpyrrolidine, N-dodecylpiperidine, N-dodecylhomopiperidine, and combinations thereof.

68. The formulation of claim 39, further comprising at least one additional active agent.
